(19) United States
(12) Patent Application Publication
(10) Pub. No.: US 2006/0182720 A1
(43) Pub. Date: Aug. 17, 2006

(54) Cell preparation and use of the preparation for treating joints and cartilage defects, and methods for the production thereof

(76) Inventor: David Wohlrab, HALLE (DE)

Correspondence Address: NOVAK DRUCE DELUCA & QUIGG, LLP, 1300 EYE STREET NW, SUITE 400 EAST TOWER, WASHINGTON, DC, 20005, US

(21) Appl. No.: 10/549,684

(22) PCT Filed: Mar. 18, 2004
(86) PCT No.: PCT/EP04/02820

§ 371 Date: Sep. 19, 2005

(30) Foreign Application Priority Data
Mar. 18, 2003 (DE) 103 11 889.6

(51) Int. Cl. A61K 35/12

(52) U.S. Cl. 424/93.7

(57) ABSTRACT

The invention relates to a cell preparation for therapeutic and/or cosmetic use in humans and/or animals, containing: a) human or animal cells, which; b) had been cultured while using a substance or substance mixtures that activates) CD44 expression of these cells, and which present an increased CD44 expression of these cells.

The invention relates to new intraarticularly, intradiscally, subcutaneously, intracutaneously or epicutaneously (topically) applicable cell preparations, which contain human or animal cells and were cultivated using a substance or mixtures of substances which activate(s) the CD44 expression of these cells, the preparation having an increased CD44 expression of these cells. Hyaluronic acid is then bonded to the thus pre-treated cells.

These cell preparations serve for medical treatment and/or cosmetic application. The invention relates furthermore to a method for producing the preparation.

Arthrosis begins with initial damage to the cartilage tissue because of various causes. There thereby occurs, in addition to a reduction in the number of intact chondrocytes, reactive synovialitis which for its part leads both to pathological changes in the synovial fluid, inter alia to a reduction in the concentration and the molecular weight of the hyaluronic acid, and also the release of inflammation mediators. This leads to secondary cartilage damage and hence finally to arthrosis which, in addition to cartilage tissue, affects all other articular structures. It is known that intraarticularly applied hyaluronic acid leads to improvement in joint mobility, to pain reduction, to inhibition of the inflammatory processes and, under in vitro conditions, to an increase in chondrocyte proliferation (K. Kawasaki et al. (1999) Hyaluronic acid enhances proliferation and chondroitin sulfate synthesis in cultured chondrocytes embedded in collagen gels. J Cell Physiol. 179: 142-148; D. Wohlrab et al. (2000) Differences in the reaction of human chondrocytes to different hyaluronic acid preparations. Hylan news. 2: 2-5).

The therapeutic effectiveness of intraarticularly applied hyaluronic acid is dependent upon a plurality of factors. Due to the considerable molecular size of hyaluronic acid (1-6×10⁶ Da), this must be split several times before it can leave the intraarticular space and be decomposed or incorporated in cartilage tissue. These splitting processes take hours up to several days dependent upon the mol mass of the hyaluronic acid. This process is also controlled substantially by the ability of chondrocytes to bond hyaluronic acid via receptors, as a result of which an extended intraarticular dwell time can be demonstrated for hyaluronic acid in comparison to other, low molecular substances (such as e.g. local anaesthetics).

The matrix receptors CD44 and/or integrins are expressed on the surface of the cell membrane of chondrocytes. Both receptor families play a central role in the homeostasis of the articular cartilage. In particular, the CD44 receptor, a very variable and multifunctional glycoprotein, was recognised as the most important cell surface receptor for hyaluronic acid (Proteins 39 (2000) 103-111). The CD44 receptor is located on the surface of the cell membrane of many vital human cells. It is a hyaluronic acid-bonding, membrane-intercalating glycoprotein which interacts with cytoskeletal proteins. It is involved both in intracellular signal transmission and in a cell-cell or cell-matrix interaction. The precise mechanism of these processes is to date not completely known. CD44 has a high affinity for hyaluronic acid. Lesser affinities are present also for chondroitin sulphate and heparan sulphane.

The CD44 gene is located on chromosome 11. It comprises 20 exons, of which 10 are always expressed (CD44H). The other 10 code for extracellular regions (called v1-v10). These are designated as different splice variants. CD44 isoforms have been detected in various human tissue types (e.g. tonsils, thyroid, breast, prostate, cervix, oesophagus, epithelium, skin).

The CD44 receptor has already been detected on the cell membrane of human chondrocytes. The CD44 expression of the chondrocytes in hyaline cartilage tissue is an essential prerequisite for the bonding of hyaluronic acid, the intensity of the hyaluronic acid bonding being dependent upon the number, the activation and intensity of the expression of corresponding receptors. The hyaluronic acid bonded in the cartilage tissue is able to fulfil the corresponding functions as matrix building block.

The chemical structure of hyaluronic acid (hyaluronan) corresponds to the formula

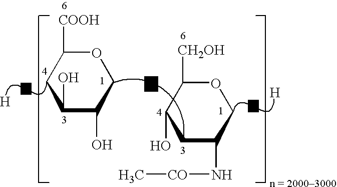

Despite the positive clinical experiences with high molecular hyaluronic acid or the salts thereof (mol mass>1×10⁶ Dalton), knowledge concerning the operating mechanism during maintenance of the homeostasis of the articular cartilage is incomplete. The current state of knowledge identifies intraarticularly applied hyaluronic acid as a lubricant and sliding agent.

It has furthermore been detected that hyaluronic acid has intraarticularly anti-inflammatory properties. The current results which have been achieved with high molecular hyaluronic acid or the salts thereof are however unsatisfactory with respect to regeneration of the articular structures.

Hence it was the object to make available intraarticularly, intradiscally, subcutaneously or intracutaneously applicable cell preparations, in order to make possible an improved regeneration of articular structures, in particular of the articular cartilage and/or articular function and/or inflammation inhibition, by means of hyaluronic acid and/or the salts thereof, compared to the unaccompanied substitution of hyaluronic acid. A further object resides in indicating a suitable method for producing a preparation of this type.

The object is achieved with respect to the cell preparation by the features of patent claim 1 and with respect to the use by the features of claim 15. The method for producing the cell preparation is characterised by the features of claims 19 to 21. The sub-claims indicate advantageous developments.

According to the invention, a cell preparation for therapeutic and/or cosmetic application in humans and/or animals is hence proposed, which preparation contains human or animal cells which were cultivated using a substance or mixture of substances which activate(s) the CD44 expression of these cells, the cell preparation having an increased CD44 expression of these cells. This cell preparation displays outstanding bonding properties for hyaluronic acid, the salts thereof and/or fragments.

Surprisingly, it was shown that a cell preparation of this type which can be applied intraarticularly, intradiscally, subcutaneously or intracutaneously shows a significantly improved regeneration of articular structures compared to hyaluronic acid and/or the salts thereof.

For the cell preparation according to the invention basically all human or animal cells are suitable. Preferably the cells are of autologous, allogeneic or xenogeneic origin. The inventor could show that it was thereby preferable if the cells are chondrocytes, keratinocytes, fibroblasts and/or meniscus cells. The used cells can be isolated from tissue or be obtained by differentiation processes from other cell systems, e.g. stem cells or fibroblasts.

It is thereby particularly preferred if in the case of chondrocytes these were isolated from the cartilage tissue. In the case of keratinocytes, it is convenient if these are isolated from the epidermis. If meniscus cells are used, these can be isolated from the meniscus tissue. Obviously the previously described cells can also be obtained by differentiation processes from other cell systems e.g. from stem cells or fibroblasts.

According to the invention, the invention includes, in the case of hyaluronic acid, also the physiologically compatible salts thereof and fragments of these compounds. The invention obviously includes also embodiments in which hyaluronic acid and the fragments are used together.

It has furthermore been shown that it is advantageous if the proportion of the compound or compounds selected from hyaluronic acid, the physiologically compatible salts of hyaluronic acid and the fragments of these compounds is from 0.001 to 5.0% by weight relative to the total galenic formulation.

In the case of the cell preparation according to the invention, it is preferred if the cells are chondroitin. Obviously the cell preparation can contain in addition also chondroitin sulphate and fragments of these compounds. A further preferred embodiment provides that in the cell preparation one or more components of the physiological cartilage (for example glycosaminoglycan or proteoglycan) are contained. It is also preferred if one or more substances with radical interceptor properties are contained. Examples thereof are tocopherol or flavonoids. Further substances which the cell preparation can contain are one or more substances with a steroidal or corticosteroidal effect, one or more non-steroidal antiphlogistics (also non-steroidal antirheumatics), one or more analgesics, one or more substances with an inhibitory effect on prostaglandin synthesis, in particular lipoxygenase inhibitors, cyclo-oxygenase inhibitors, cyclo-oxygenase inhibitors and phospholipase A2 inhibitors, one or more growth-stimulating or growth-regulating substances, so-called growth factors, one or more vitamins, one or more antioxidants and/or one or more substances with water-binding properties.

Preferably, at least one local anaesthetic or derivatives of this compound is used as the substance activating the CD44 expression of cells, in particular chondrocytes.

The cell preparation according to the invention can per se be made available in all known current formulations. Hence it is preferred to apply the cell preparation in the form of a matrix, a solution, a suspension, an emulsion, a paste, a salve, a gel, a cream or a lotion or a spray.

The invention relates furthermore to the use of the previously described cell preparation for therapeutic and/or cosmetic application in humans and/or animals.

The invention also relates to the use of the previously described cell preparation for producing a pharmaceutical, in particular for producing a pharmaceutical for intraarticular, intradiscal, subcutaneous, intracutaneous or epicutaneous (topical) application.

It is thereby preferred if the cell preparation is applied for the treatment of degenerative diseases of human or animal joints.

Some specific uses of selected cell preparations are indicated subsequently.
The use of one or more compounds, selected from human chondrocytes or other human or animal cells, hyaluronic acid, the physiologically compatible salts of hyaluronic acid and the fragments of these compounds and a substance or mixtures of substances which activate(s) the CD44 expression of chondrocytes, preferably a local anaesthetic or a plurality of local anaesthetics or the derivatives of these compounds, in order to produce an intraarticularly, intradiscally, subcutaneously or intracutaneously applicable cell preparation,
the use of one or more compounds, selected from human chondrocytes or other human or animal cells, hyaluronic acid, the physiologically compatible salts of hyaluronic acid and fragments of these compounds and of a substance or mixture of substances which activate(s) the CD44 expression of chondrocytes, in order to increase the human and/or animal chondrocyte proliferation,
the use of one or more compounds, selected from human chondrocytes or other human or animal cells, hyaluronic acid, the physiologically compatible salts of hyaluronic acid and fragments of these compounds and of a local anaesthetic or a plurality of local anaesthetics or the derivatives of these compounds, in order to change the CD44 receptor expression,
the use of one or more compounds, selected from human chondrocytes or other human or animal cells, hyaluronic acid, the physiologically compatible salts of hyaluronic acid and fragments of these compounds and of a local anaesthetic or a plurality of local anaesthetics or the derivatives of these compounds, in order to stabilise and/or regenerate individual or a plurality of articular structures, in particular the articular cartilage and menisci,
the use of one or more compounds, selected from human chondrocytes or other human or animal cells, hyaluronic acid, the physiologically compatible salts of hyaluronic acid and fragments of these compounds and of a local anaesthetic or a plurality of local anaesthetics or the derivatives of these compounds, in order to improve joint mobility,
the use of one or more compounds, selected from human chondrocytes or other human or animal cells, hyaluronic acid, the physiologically compatible salts of hyaluronic acid and fragments of these compounds and of a local anaesthetic or a plurality of local anaesthetics or the derivatives of these compounds, in order to inhibit inflammatory processes,
the use of one or more compounds, selected from human chondrocytes or other human or animal cells, hyaluronic acid, the physiologically compatible salts of hyaluronic acid and fragments of these compounds and of a local anaesthetic or a plurality of local anaesthetics or the derivatives of these compounds, in order to improve pain reduction.

The application of the cell preparation according to the invention can be effected both on humans and on animals. The cell preparations according to the invention can be used both in human and in veterinary medicine. The application fields of the cell preparations according to the invention relate to the therapy, prophylaxis and/or metaphylaxis of articular cartilage, bone and cartilage bone defects, meniscus and intervertebral disc lesions and also other cartilage defects (e.g. nose and ear cartilage) also from a cosmetic point of view.

The invention relates furthermore to a method for producing the previously described cell preparation. According to the invention, human or animal cells are thereafter cultivated, using a substance or mixture of substances which activate(s) the CD44 expression of these cells, from said substance or mixture of substances. It is now essential that the CD44 receptor expression is increased superproportionally only for a specific time. It has been shown that in the case of a timespan for the cultivation of the cells of 6 to 15 days, preferably 9 to 11 days, most particularly preferred after 10 days, the CD44 receptor expression is at its best within 12 to 72 hours. Within this timespan, 24 to 48 hours are particularly favourable. During this timespan, the cells have obviously particularly plentiful free receptors for CD44, so that they bond well to the hyaluronic acid.

If necessary, further auxiliary active substances and/or carrier materials can be added in order to obtain a suitable formulation. Subsequently suitable methods are indicated.
Production of an intraarticularly, intradiscally, subcutaneously or intracutaneously applicable cell preparation, wherein human chondrocytes or other human or animal cells, one or more compounds selected from hyaluronic acid, the physiologically compatible salts of hyaluronic acid and the fragments of these compounds and a substance or mixtures of substances which activate(s) the CD44 expression of chondrocytes, if necessary with further active substances, auxiliary and/or carrier materials, are put into a suitable cell preparation.
Production of an intraarticularly, intradiscally, subcutaneously or intracutaneously applicable cell preparation, wherein there are achieved human chondrocytes or other human or animal cells, one or more compounds selected from hyaluronic acid, the physiologically compatible salts of hyaluronic acid and the fragments of these compounds, and a substance or mixtures of substances which activate(s) the CD44 expression, and via the pH value of the formulation an optimal bonding of the cells to hyaluronic acid or of the hyaluronic acid to the cells and/or the physiologically compatible salts of hyaluronic acid and/or the fragments of these compounds.

The invention is intended to be explained with reference to a concrete example, without being restricted thereto:

### EXAMPLE 1

Influencing the CD44 Receptor Expression of Human Chondrocytes by Means of Lidocaine

Production

Lidocaine hydrochloride (University pharmacy of the Martin Luther University Halle-Wittenberg) was present primarily in powder form. This was dissolved in a corresponding quantity in RPMI medium (Seromed, Berlin) so that an end concentration of 0.1 mmol/l lidocaine was present. Sterile filtration was effected subsequently. The addition of substance to the cell culture was effected during the last medium change on the 10th culture day.

Preparation of the Biological Material

The tests were effected on human chondrocytes which were isolated from arthrotically changed knee joint cartilage. The cartilage tissue stemmed from femoral articular surfaces resected during implantation of total knee endoprostheses. Exclusively arthrotically changed cartilage tissue from three different patients without known relevant secondary diseases, in particular without rheumatoid arthritis, was used.

The intraoperatively obtained bone-cartilage fragments were transferred firstly into sterile L15 medium (Seromed, Berlin) as transport medium. Subsequently, the separation of the cartilage tissue from the subchondral bone was effected under sterile conditions by means of a scalpel and also sharp severance of the tissue into pieces of approximately 1 mm³. The enzymatic isolation of the chondrocytes from the pieces of cartilage was effected by means of pronase and collagenase A (Boehringer Mannheim) over a timespan of 16 hours.

Test Conditions

The isolated chondrocytes were cultivated in cell culture flasks in RPMI medium (Seromed, Berlin) with the addition of various antibiotics at 37° C. and 5% carbon dioxide in an incubator as a monolayer culture. The medium change was effected every 2 days. On the 10^{th} culture day, a medium change was effected for the last time. Here the addition of lidocaine in the cell culture medium was effected in a concentration of 0.1 mmol/l. In addition, an untreated chondrocyte population respectively was run jointly as control. The culture duration was 11, 12 or 13 days.

Implementation of the Test

The detection of the CD44 membrane protein in the case of human chondrocytes was effected 24, 48 and 72 hours respectively after addition of the lidocaine in a flow cytometrical manner by comparison with the isotypical control (ms-IgG1-FITC, Mouse IgG1, DAKO Diagnostika GmbH, Mannheim). The number of feature-carrying cells was thereby plotted against the fluorescence intensity of the FITC-conjugated CD44 antibody (anti-CD44-FITC, DAKO).

The results of the tests are represented in FIG. 1

[image]

1. Cell preparation for therapeutic and/or cosmetic application in humans and/or animals, containing human or animal cells which were cultivated using a substance or mixtures of substances which activate(s) the CD44 expression of these cells and to which cells hyaluronic acid and/or the physiologically compatible salts thereof and/or fragments herefrom are bonded.

2. Cell preparation according to claim 1, characterized in that the proportion of the compound or compounds selected from hyaluronic acid, the physiologically compatible salts of hyaluronic acid and the fragments of these compounds is from 0.001 to 5.0% by weight relative to the total formulation.

3. Cell preparation according to claim 1, characterized in that the substance which activates the CD44 expression of the cells is at least a local anaesthetic and/or a derivative and/or a mixture herefrom.

4. Cell preparation according to claim 1, characterized in that the used cells are of autologous, allogeneic or xenogeneic origin.

5. Cell preparation according to claim 1, characterized in that the cells are chondrocytes, keratinocytes, fibroblasts and/or meniscus cells.

6. Cell preparation according to claim 1, characterized in that the cells are isolated from tissue or have originated from other cell systems by differentiation processes.

7. Cell preparation according to claim 5, characterized in that chondrocytes are contained which were isolated from the cartilage tissue or have originated from other cell systems by differentiation processes.

8. Cell preparation according to claim 5, characterized in that keratinocytes are contained which were isolated from the epidermis or have originated from other cell systems by differentiation processes.

9. Cell preparation according to claim 5, characterized in that fibroblasts are contained which were isolated or have originated from other cell systems by differentiation processes.

10. Cell preparation according to claim 5, characterized in that meniscus cells are contained which were isolated from the meniscus tissue or have originated from other cell systems by differentiation processes.

11. Cell preparation according to claim 1, characterized in that, in addition, chondroitin, chondroitin sulphates and fragments are contained in these compounds.

12. Cell preparation according to claim 1, characterized in that, in addition, one or more compounds of the physiological cartilage are contained.

13. Cell preparation according to claim 1, characterized in that, in addition, one or more substances with radical interceptor properties, one or more substances with a steroidal or corticosteroidal effect, one or more non-steroidal antiphlogistics one or more analgesics, one or more substances with an inhibitory effect on prostaglandin synthesis, in particular lipoxygenase inhibitors, cyclo-oxygenase inhibitors and phospholipase A2 inhibitors, one or more growth-stimulating or growth-regulating substances (so-called growth factors), one or more vitamins, one or more antioxidants and/or one or more substances with water-binding properties are contained.

14. Cell preparation according to claim 1, characterized in that it is preferably present in the form of a matrix, a solution, a suspension, an emulsion, a paste, a salve, a gel, a cream, a lotion or a spray.

15. (canceled)

16. A pharmaceutical preparation comprising the cell preparation according to claim 1 and further auxiliary active substances or carrier materials.

17. An intraarticularly, intradiscally, subcutaneously, intracutaneously or epicutaneoulsy (topically) applicable transplantate, comprising the cell preparation according to claim 1.

18. A method for the treatment of the degenerative diseases of human or animal joints, for the therapy, prophylaxis and metaphylaxis of articular diseases and of articular function defects, for the therapy, prophylaxis and/or metaphylaxis of articular cartilage and cartilage bone defects, for the therapy, prophylaxis and/or metaphylaxis of meniscus and intervertebral disc diseases, for the cosmetic and/or therapeutic treatment of extraarticular cartilage defects and/or for the therapy, prophylaxis and/or metaphylaxis of tissue defects of the skin organ which comprises treating a human or animal with an effective amount of the cell preparation according to claim 1.

19. Method for producing a cell preparation according to claim 1, which comprises cultivating human or animal cells, using a substance or mixtures of substances which activate(s) the CD44 expression of these cells, and by bonding hyaluronic acid and/or the physiologically compatible salts thereof and/or fragments herefrom to these thus pre-treated cells.

20. Method according to claim 19, characterized in that the cultivation of the cells is effected over a timespan of 6 to 15 days, and then the substance which activates the CD44 expression is set.

21. Method according to claim 20, characterized in that the hyaluronic acid and/or the salts thereof and/or the fragments thereof are bonded 12 to 72 hours after addition of the substance which activates the CD44 expression of the cells.
